(19) United States
(12) Patent Application Publication
(10) Pub. No.: US 2008/0009762 A1
(43) Pub. Date: Jan. 10, 2008

(54) Method and apparatus for interpreting capnographic data

(75) Inventors: David R. Hampton, Woodinville, WA (US); Baruch S. Krauss, Brookline, MA (US); Paul W. Schmitt, Woodinville, WA (US)

Correspondence Address: MARY Y. REDMAN;MEDTRONIC, INC., 710 MEDTRONIC PARKWAY NE M.S. LC340, MINNEAPOLIS, MN, 55432-5604, US

(73) Assignee: Medtronic Emergency Response Systems, Inc.

(21) Appl. No.: 11/588,990

(22) Filed: Oct. 27, 2006

Related U.S. Application Data
(60) Provisional application No. 60/817,175, filed on Jun. 27, 2006.

(51) Int. Cl. A61B 5/08

(52) U.S. Cl. 600/532

(57) ABSTRACT

A method for interpreting physiological data includes obtaining a capnogram; obtaining a plurality of data points from the expiratory rise segment and the alveolar plateau segment of the capnogram; fitting a non-linear function to the plurality of data points; and calculating a plurality of calculated parameters that characterize the non-linear function. The obtaining of data point, fitting of a non-linear function, and calculating parameters may be performed for two or more successive capnogram waveforms. The non-linear function may be a cumulative Weibull function.

### BACKGROUND OF THE INVENTION

1. Field of the Invention

The present invention relates to the field of capnography in general, and in particular to analysis and interpretation of the waveforms obtained in capnographic measurements.

### TECHNICAL BACKGROUND

Capnography is the non-invasive measurement of the concentration of carbon dioxide in a subject's breath. Capnography is routinely used for monitoring a patient's ventilatory status where the capnogram, a graphical representation of carbon dioxide concentration as a function of time, is displayed on a monitor and interpreted by visual inspection.

Visual evaluation and interpretation of the capnogram is typically done by qualitative pattern recognition. Pattern recognition is a technique in which a capnogram is visually compared to a set of abnormal capnograms, with a corresponding differential diagnosis for each abnormal shape. This requires considerable skill and specialized knowledge on the part of the person doing the visual comparison, and is useful only for conditions or diseases that result in a large-scale abnormality in capnogram shape that is clearly visible to the naked eye.

### SUMMARY OF THE INVENTION

In one aspect, the invention provides a method for interpreting physiological data which includes the following steps: obtaining a capnogram; obtaining a plurality of data points from the expiratory rise segment and the alveolar plateau segment of the capnogram; fitting a non-linear function to the plurality of data points; and calculating a plurality of calculated parameters that characterize the non-linear function. In one embodiment, the obtaining of data point, fitting of a non-linear function, and calculating parameters may be performed for two or more successive capnograms.

In another aspect, the invention provides a method for interpreting physiological data that includes the above steps and wherein the non-linear function is a cumulative Weibull function.

In another aspect, the invention provides a method for interpreting physiological data which includes the following steps: obtaining a capnogram; obtaining a plurality of data points from the expiratory rise segment and the alveolar plateau segment of the capnogram; fitting a non-linear function to the plurality of data points; calculating a plurality of calculated parameters that characterize the non-linear function; and comparing at least one of the plurality of calculated parameters to at least one standard parameter value.

In another aspect, the invention provides a method for interpreting physiological data which includes the following steps: obtaining a capnogram; obtaining a plurality of data points from the expiratory rise segment and the alveolar plateau segment of the capnogram; fitting a non-linear function to the plurality of data points; calculating a plurality of calculated parameters that characterize the non-linear function; and comparing at least one of the plurality of calculated parameters to at least one standard parameter value; and presenting an aural or visual representation of the output of the comparing step. The representation may be, for example, a visual or aural alarm if the parameter is outside certain limits, or a visual or aural notification of a probable diagnosis, or a visual or aural notification of a recommended therapy.

In another aspect, the invention provides a method for interpreting physiological data which includes the following steps: obtaining a capnogram; obtaining a plurality of data points from the expiratory rise segment and the alveolar plateau segment of the capnogram; fitting a non-linear function to the plurality of data points; and calculating a plurality of calculated parameters that characterize the non-linear function; obtaining other physiological data in addition to capnogram data; analyzing the other data; and determining a patient condition based on a combination of the output of the step of analyzing said other data step and the step of calculating parameters. The other data may include, for example, at least one of respiration rate, inhalation time, exhalation time, EtCO₂, and PaCO₂.

In another aspect, the invention provides a method for interpreting physiological data which includes the following steps: obtaining a capnogram; obtaining a plurality of data points from the expiratory rise segment and the alveolar plateau segment of the capnogram; fitting a non-linear function to the plurality of data points; calculating a plurality of calculated parameters that characterize the non-linear function; and categorizing the capnogram on the basis of the calculated parameters.

An advantage of embodiments of the present invention is the provision of a method and device for discerning small, yet diagnostically significant, changes in the shapes of segments of the capnogram. The ability to discern minor gradations in shape of capnogram, makes it possible to use capnographic data to evaluate evolving trends in a patient's condition, which are not discernable by conventional visual inspection.

Another advantage of embodiments of the invention is the ability to perform quantitative analysis and characterization of the capnogram. This results in the capnogram having considerable precision as a diagnostic tool. This also makes possible the use of capnographic data to monitor the effect of therapy or medical procedures on a patient. Because, in an embodiment of the invention, quantitative analysis may be performed automatically by a microprocessor, capnographic data can be used as a diagnostic tool even by individuals who do not have the specialized knowledge and skill needed for conventional visual analysis.

It is to be understood that both the foregoing general description, and the following detailed description are exemplary and explanatory and are intended to provide further explanation of the invention as claimed. The accompanying drawings are included to provide a further understanding of the invention and are incorporated in and constitute a part of this specification, illustrate several embodiments of the invention and, together with the description, serve to explain the principles of the invention.

### BRIEF DESCRIPTION OF THE DRAWINGS

FIG. 1A is an illustration of a typical capnogram for a healthy individual;

FIG. 1B is an illustration of a typical capnogram for an individual with obstructive lung disease;

FIG. 2 is a schematic block diagram of a device according to an embodiment of the present invention;

FIG. 3 is an illustration of a capnogram;

FIGS. 4A, 4B, 4C and 4D are plots of capnographic data points with a best-fit cumulative Weibull curves superimposed thereon; and

FIGS. 5, 6, and 7 are flowcharts illustrating processes according to embodiments of the invention.

### DETAILED DESCRIPTION

Reference will now be made in detail to the present preferred embodiment of the invention, an example of which is illustrated in the accompanying drawings. Wherever possible, the same reference numbers are used throughout the drawings to refer to the same or like parts.

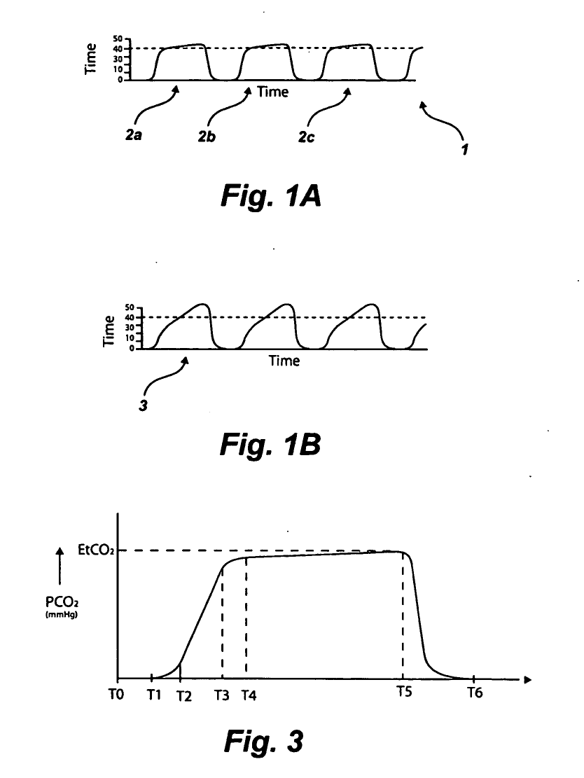

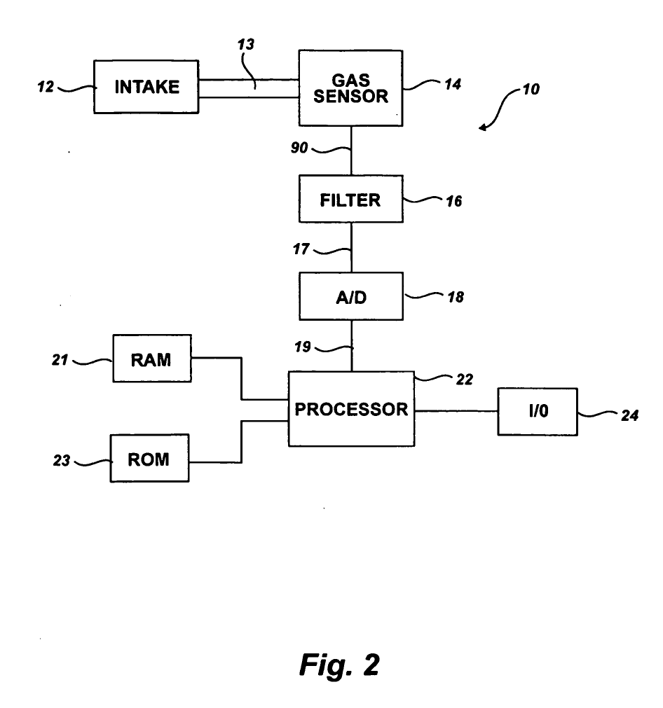

Referring to FIG. 1A, a capnogram 1 is a graphical representation of CO₂ concentration, typically expressed as partial pressures for CO₂, in a subject's breath, plotted as a function of time. This results in a series of waveforms 2a, 2b, 2c, etc., each one of which displays CO₂ concentration data for a single tidal breath. For an illustrative comparison, FIG. 1A shows a typical capnogram 1 for a healthy individual, and FIG. 1B shows a typical capnogram 3 for an individual with obstructive lung disease.

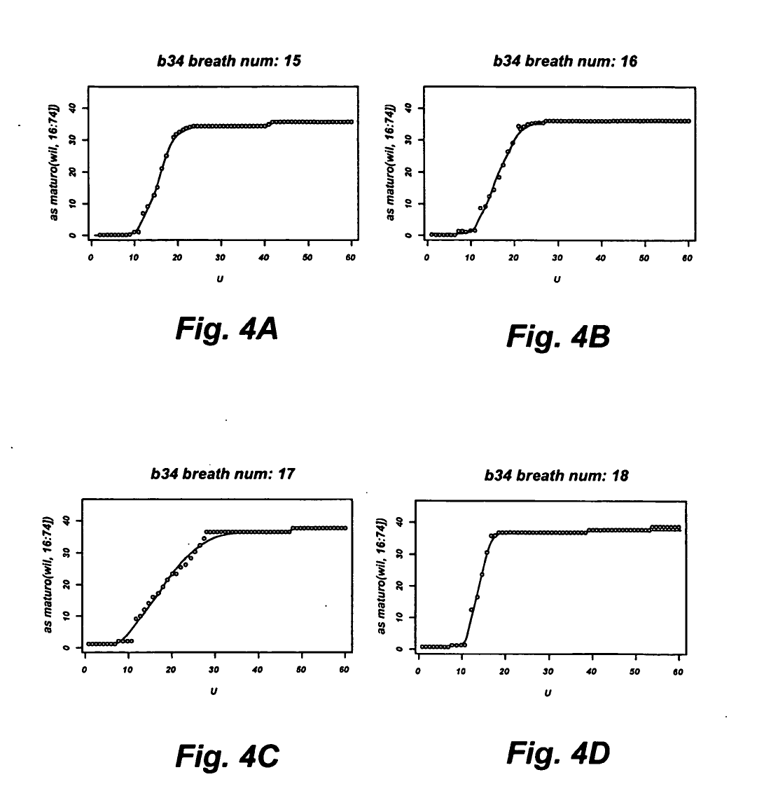

According to a first embodiment of the invention, a method for interpreting physiological data includes the step of obtaining capnographic data, preferably in the form of a capnogram. Such data may be obtained by a device known as a capnograph. Capnographs having data acquisition capabilities suitable for use in an embodiment of the invention are commercially available and include, for example, the capnograph monitor sold under the trademark “VitalCap” by Oridion Corp of Jerusalem, Israel, or the capnograph sold under the trademark “LifeCap” by Medtronic Emergency Response Systems Inc. of Redmond, Wash. USA. Reference is now made to FIG. 2, which is a schematic block diagram of a capnograph 10 capable of analyzing and interpreting capnograms obtained from a subject, according to an embodiment of the present invention. Samples of the exhaled breath of a patient are drawn through an intake 12, such as a nasal cannula, mask or the like, into a sampling tube 13 that directs the sample to a gas sensor 14 in the capnograph 10. The intake 12 preferably is a disposable interface that that can be removably coupled to the capnograph 10. The gas sensor 14 analyzes the sample for CO₂ content using techniques such as, for example, infrared detection, that track instantaneous CO₂ concentration as a function of time. Gas sensor 14 generates an electronic analog output signal corresponding to measurements of the instantaneous concentration levels of CO₂ and passes the signal to a low-pass filter 16, which prevents aliasing. Filter 16 passes filtered measurement data 17 to an analog-to-digital converter 18 where the filtered analog measurement data 17 are converted into digital measurement data 19. The digital measurement data 19 may be stored in random access memory (RAM) 21.

Analysis of the digital waveform measurement data is performed by a processor 22 in a manner described in detail below. In some embodiments, data stored in a read-only memory (ROM) 23 may be used by the processor 22 in its analysis. The processor 22 reports the results of its analysis through an input/output (I/O) device 24. I/O device 24 may be a visual display such as a text screen, LCD screen capable of displaying graphics or pictures, LED indicator lights, etc., or an aural indicator such as a speaker which outputs recorded voiced messages or sounds such as beeps, or other sound-emitting device. A user may input commands or information to the processor 22 through the I/O device 24.

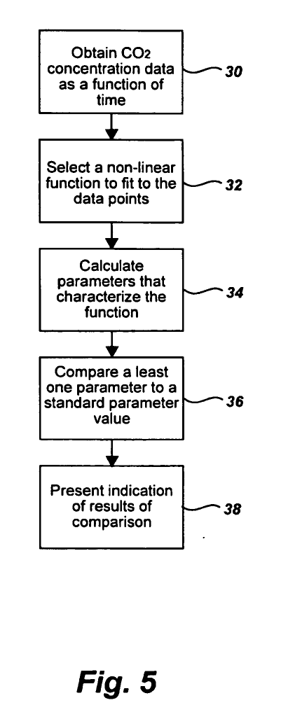

FIG. 3 is an illustration of a typical capnogram obtained from an adult with normal lung function. During inhalation, the gas that enters the gas sensor is environmental air surrounding the inlet port(s) of the intake 12 and the value of the partial pressure of carbon dioxide, PCO₂ is nearly zero. This is shown graphically by the capnograph segment from time T0 to time T1. At the start of exhalation at time T1, the concentration of CO₂ rises as gas from the ventilatory dead space is exhaled from the airway. Starting at time T2, the concentration of CO₂ rises rapidly as gas from the alveolar mixes with dead space gas. As exhalation continues, the rate of increase in CO₂ concentration begins to slow at about time T3 until at time T4 a phase of gradually rising CO₂ concentration, in which alveolar gas is exhaled, is reached. This phase lasts until time T5. This phase of the capnogram from T4 to T5 is commonly referred to as the alveolar plateau. The value of the CO₂ concentration rises gradually during the whole of the plateau, up to the end tidal value, EtCO₂ at time T5. This marks the onset of the inhalation phase, with CO₂ concentration falling back to a nearly zero value at time T6, as environmental air traverses the gas sensor 14 with continued inhalation.

Typically, the capnogram corresponding to a single tidal breath has been described as if it had four visible linear phases. Visually, each of these four phases (i.e., on the time axis, from T0 to T1; from T1 to T3 from T3 to T5, and from T5 to T6), has conventionally been approximated as a straight line. Thus, the capnogram for a single tidal breath of a subject with normal lung function has been described as a trapezoid shape. The two phases from T1 to T3 and from T3 to T5, and the two corresponding line segments of the trapezoidal model, are commonly referred to as the “Expiratory Rise” and the “Alveolar Plateau”. Since each of the two line segments in the trapezoidal model can mathematically be described by two independent variables (e.g.: the slope and intercept, or, equivalently, the angle and length), a total of four measures suffice to completely capture the information contained in the Expiratory Rise and the Alveolar Plateau in the trapezoidal approximation. Most commonly, the angle of the line, referenced to the horizontal, and the duration of the phase have been used to characterize the capnogram in this model. However, the segmentation of exhalation into two discrete, linear phases, characteristic of the trapezoidal model, is an artificial simplification, ignoring deviations from linearity within a phase and the character of turning points between phases.

The method of analysis performed by the processor 22 according to an embodiment of the invention will now be described. In an embodiment of the invention, the processor 22 performs an analysis in which it models one or more segments of a capnogram as a nonlinear function of time, using curve-fitting techniques. In an embodiment of the invention, modeling the expiratory rise and alveolar plateau phases of the capnogram using a non-linear curve fit characterized by four calculated parameters has been found to be advantageous. This results in diagnostic utility for the capnogram that heretofore was unavailable.

In a preferred embodiment, a cumulative Weibull function is used as the non-linear function which is fit to the capnographic data by curve-fitting techniques well-known to those in the art. A cumulative Weibull function has the following form:

$$y = a [ 1 - exp ( - {( \frac{x + {c ( ln 2 )}^{1 / d} - b}{c} )}^{d} ) ]$$

where transition height=a;
transition center=b; and
transition width=2^{1/d}c(In 2)^{1/d}−c(2 In 2−In 3)^{1/d},
and where c>0; d>0; x>b−c(In 2)^{1/d}

The parameters a, b, c and d can be used to characterize a particular cumulative Weibull curve fit to a particular data set.

Other non-linear functions can be used in embodiments of the present invention as well. For example, a cumulative Gaussian curve or logistic curve, can be used as the non-linear function to be fit to the data points of a particular capnogram.

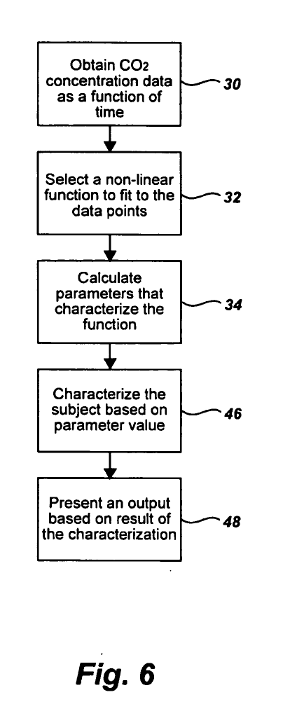

FIGS. 4A, 4B, 4C and 4D are examples of plots of capnographic data points for four successive breaths in a capnogram of an adult with normal lung function (hereinafter referred to as a normal capnogram), each with a best fit cumulative Weibull curve superimposed thereon.

This data was obtained by collecting capnographic data from a population of 30 adults with normal lung function as determined by history, physical examination, and spirometry. For each subject, a 3-minute data-gathering period was commenced when the subject was observed to be consistently executing normal tidal breathing. Data was collected continuously through the subsequent recording period, resulting in approximately 30 breaths being recorded from each subject.

At the conclusion of the recording, data were transmitted to a desktop computer for analysis. A three-phase pre-analysis procedure was performed on each subject recording to identify fiducial points and to assure waveform quality prior to modeling and measurement. These fiducial points are each defined by their sequence location and associated CO₂ value (P(time, amp)).

First, each continuous capnogram was scanned to identify transitions between inhalation and exhalation. The start of inhalation was identified using slope and amplitude criteria: a fiducial mark, P1(time, amp), was placed at the point in the capnogram where the CO₂ concentration exceeded 4 mmHg and the rising slope exceeded +xx mmHg/sec. Another fiducial point identifying the start of exhalation P3(time, amp) was placed at the location of the end-tidal CO₂ value, defined as a falling slope of more than −zz mmHg/sec and an amplitude less than 4 mmHg of the maximum value since the preceding start of inhalation.

Two additional fiducial marks were placed to mark the start of the Alveolar Plateau (P2(time, amp)) and the return to baseline on inhalation (P4)time, amp)). P2 was established at the crossing point of two regression lines fit through the Expiratory Rise and alveolar plateau, generally meeting above the “knee” of the ascending capnogram. P4 was defined as the point at which the CO₂ value fell below 4 mmHg in association with a falling slope of more than −yy mmHg/sec.

Finally, the annotated record was scanned to eliminate artifacts caused by coughing, sighing, and other normal physiologic and behavioral variants.

Non-linear functions suitable for characterizing the capnogram were determined through a statistical curve-fitting procedure. All normal breaths were extracted, superimposed, and aligned on their P1 fiducial marker. A median waveform was created from the population by taking the point-by-point median values at each sample point from (P1−10) to (P1+70), which included two seconds of exhalation. An automated curve-fitting system, the TableCurve2D v5.01 software program sold by Jandel Scientific of San Rafael, Calif., was used to fit candidate functions to the median capnogram, and to rank them in order of goodness-of-fit. The cumulative Weibull distribution function was selected as the best four-parameter approximation to the exhalation capnogram and is described by parameters a and b, defining the height and centering of the inflection point of the rising phase of the curve, and c and d, which define the width and trajectory of the takeoff and plateau transitions.

It will be readily understood that parameters for cumulative Weibull curves for representative or standard capnograms for various disease states and other abnormal lung function states can be determined by sampling subjects with the particular disease states or abnormal lung function desired to be studied, and fitting a cumulative Weibull function to the median waveform using techniques similar to those described above.

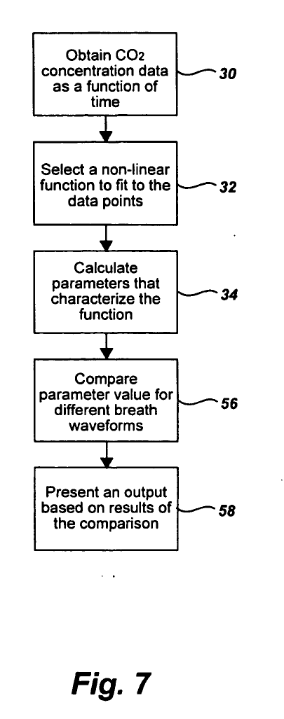

FIGS. 5, 6, and 7 are flow charts of processes according to several illustrative embodiments. In the embodiments of FIGS. 5, 6 and 7, in a step 30 of the process, data is collected on CO₂ concentration in a subject's breath as a function of time by, for example, a capnogram as illustrated in FIG. 2. The data points in this data set make up sequential waveforms in the subject's capnogram. In a second step 32, a non-linear function, such as a cumulative Weibull curve, is selected to be fit to a segment (preferably, a segment which includes the expiratory rise and the alveolar plateau) of a waveform derived from the data by well-known data processing techniques. For example, the waveform may be a representative waveform chosen from among the various waveforms of the capnogram. In an alternative example, the derived waveform may be a median waveform, derived by calculating the median value for each point or several chosen points in time of a number of waveforms in the capnogram. In a third step 34, parameters characterizing the best-fit curve are calculated by well-known mathematical techniques. In the embodiment of FIG. 5, in the next illustrated step 36, at least one of the resulting calculated parameters values is compared to a corresponding at least one standard parameter value. The standard parameter value may be the parameter value characterizing a standard capnogram representing normal lung function, or it may be a parameter value characterizing a capnogram representative of a disease state, or of a normal response to a therapy, or of an exertion performed by a subject having normal lung function, or any other permanent or transitory physical condition which may affect CO₂ concentration in the subject's breath. The standard parameter values are preferably stored in ROM 23 (see FIG. 2). The result of the comparison can be used as a quantitative diagnostic tool for lung function. In step 38, an output based on the result of the comparison is presented. This output can be visual, such as a text message, a symbol or a picture on an LCD display, or indicator lights, or it can be aural, such as recorded voice message, a beep, tone, or the like. The information conveyed by the output can include instruction or guidance for the user, a diagnosis, a therapy recommendation, a numerical value or other information that can be used in diagnosing, making a therapy decision, or evaluating a subject.

In the embodiment of FIG. 6, values of one or more of the calculated parameters are used to characterize the subject capnogram in a step 46. An output based on the result of this characterization is presented to the user in a step 48, in a manner similar to the output step 38 of FIG. 5.

In the embodiment of FIG. 7, in step 56, parameters for successive tidal breath waveforms (which may or may not be adjacent each other in time) in the same capnogram under study are compared to each other. In this manner, trends in respiratory function can be ascertained and quantified. This embodiment is advantageous in situations where one wishes to track a patient's condition as time passes. This embodiment may also be used to ascertain the effect on respiratory function of a therapy applied to a patient by comparing data before and after therapy is administered, or of an environmental condition to which the subject is exposed, or to a change in the bodily condition of the subject. This change in bodily condition could be, for example, a positional change, or an exercise or exertion performed on or by the subject. The ability to quantify trends and changes in respiratory function makes the embodiment useful for monitoring effect of exercise or of therapies on respiratory function or change in the physical condition of the subject. Similar to the embodiments of FIGS. 5 and 6, an output based on the result of the comparison is presented in a step 58.

Calculated parameters characterizing the cumulative Weibull or other non-linear function modeling a capnogram can also be used in other manners to provide a diagnostic aid or therapeutic feedback. For example, one or more of the parameters can be examined to see if it is outside a given range of values. An output in the form of a visual or an audible indicator or alarm can be generated to alert a device user of an out-of-range capnogram parameter.

Results of the comparison step may be used to initiate a therapy or a process performed by an associated device. For example, a nebulizer may be provided with a controller that receives the result of the comparison step and activates the nebulizer therapy when a particular comparison step result is received.

The parameters of the non-linear function may be analyzed by the processor 22 in combination with other data contained in or derivable from the capnographic data, such as respiratory rate, inhalation or exhalation time, or EtCO₂, or data from other physiologic sensors, such as SpO₂, to provide additional diagnostic utility.

It will be apparent to those skilled in the art that various modifications and variations can be made to the above-described embodiment(s) of the invention without departing from the spirit and scope of the invention. Thus, it is intended that the present invention cover the modifications and variations of the embodiments provided they come within the scope of the appended claims and their equivalents.

What is claimed is:

1. A method for interpreting physiological data comprising the steps of:
a. obtaining a capnogram;
b. obtaining a plurality of data points from a segment of the capnogram;
c. fitting a non-linear function to the plurality of data points; and
d. calculating a plurality of calculated parameters that characterize the non-linear function.

2. The method of claim 1 wherein the non-linear function includes a cumulative Weibull function.

3. The method of claim 1 wherein the non-linear function includes a Cumulative Gaussian Distribution function.

4. The method of claim 1 further comprising the step of comparing at least one of the plurality of calculated parameters to at least one standard parameter value.

5. The method of claim 4 further comprising presenting a visual or aural output based on the result of the comparing step.

6. The method of claim 5 wherein the visual or aural representation includes an alarm if at least of the plurality of calculated parameters lies outside predetermined limits.

7. The method of claim 5 wherein the visual or aural output includes a notification of a probable diagnosis

8. The method of claim 5 wherein the visual or aural output includes a notification of a recommended therapy.

9. The method of claim 5 wherein the visual or aural output includes a notification of a patient response to a therapy.

10. The method of claim 5 wherein the visual or aural output includes a notification of patient response to exercise performed by the patient.

11. The method of claim 2 wherein the plurality of calculated parameters includes four calculated parameters.

12. The method of claim 1 further comprising
obtaining other physiological data in addition to capnogram data;
analyzing said other data; and
determining a patient condition based on a combination of the output of the step of analyzing said other data step and the step of calculating parameters.

13. The method of claim 12 wherein said other data includes at least one of respiration rate, inhalation time, exhalation time, EtCO₂, and PaCO₂, and SpO₂.

14. The method of claim 1 further comprising categorizing the capnogram on the basis of said calculated parameters.

15. The method of claim 1 further comprising repeating steps a, b, c, and d for a plurality of tidal breath cycles in the capnogram.

16. The method of claim 1 wherein the segment includes the expiratory rise and the alveolar plateau of the capnogram.
